(19) United States
(12) Patent Application Publication
(10) Pub. No.: US 2025/0303147 A1
(43) Pub. Date: Oct. 2, 2025

(54) NEUROSTIMULATION CATHETER

(71) Applicant: B. Braun Melsungen AG, Melsungen (DE)

(72) Inventor: Jens Wildhagen, Hannover (DE)

(21) Appl. No.: 19/089,267

(22) Filed: Mar. 25, 2025

(30) Foreign Application Priority Data
Mar. 27, 2024 (DE) 10 2024 108 690.4

(51) Int. Cl. A61N 1/05; A61M 25/04

(52) U.S. Cl. CPC A61N 1/0558; A61M 25/04

(57) ABSTRACT

A neurostimulation catheter for use in pain therapy includes a longitudinally extended catheter shaft and at least one stimulation electrode arranged on the catheter shaft and configured to deliver an electrical stimulation signal. An anchor element is secured to the catheter shaft and configured to be anchored to a body tissue that surrounds the catheter shaft. At least a portion of the anchor element is manufactured from a galvanic material that is galvanically dissolvable under the action of a DC voltage signal to release the anchor element from the body tissue.

### CROSS-REFERENCE TO RELATED APPLICATION

This application claims priority under 35 U.S.C. § 119 to German Application No. 10 2024 108 690.4, filed on Mar. 27, 2024, the content of which is incorporated by reference herein in its entirety.

### FIELD

The present disclosure relates to a neurostimulation catheter for use in pain therapy, having a longitudinally extended catheter shaft and at least one stimulation electrode that is arranged on the catheter shaft and configured to deliver an electrical stimulation signal.

### BACKGROUND

A neurostimulation catheter is known from the prior art and provided for use in pain therapy by means of electrical neurostimulation. In this case, the longitudinally extended catheter shaft is inserted into the body tissue of a patient, with the electrode arranged on the catheter shaft being positioned in the region of a nerve to be stimulated. An electrical stimulation signal is emitted by means of the electrode in order to inhibit the transmission of excitation of the nerve. The inhibited transmission of excitation alleviates or suppresses the patient's sense of pain. In this context, the distance between electrode and nerve has a decisive influence on the effectiveness of pain suppression.

### SUMMARY

It is an object of the present disclosure to provide a neurostimulation catheter of the type set forth at the outset, which allows improved pain therapy.

This problem is solved by virtue of at least one anchor element being secured to the catheter shaft, the anchor element being configured to be anchored to a body tissue that surrounds the catheter shaft, with at least portions of the anchor element being manufactured from a galvanic material, whereby at least portions of the anchor element are galvanically dissolvable under the action of a DC voltage signal, and hence the anchorage to the body tissue can be released. The solution according to the present disclosure enables an easily releasable anchorage of the catheter shaft to the patient's body tissue. The anchorage counteracts unwanted dislocations of the catheter shaft and hence unwanted changes in the distance between the electrode and the nerve to be numbed. This prevents impairment of the effectiveness of the pain therapy. The at least one anchor element is present to this end. In order to make the release of the anchorage as simple as possible, at least portions of the anchor element are manufactured from the galvanic material. The galvanic material can be (galvanically) dissolved under the action of the DC voltage signal. The anchorage to the body tissue is releasable as a result of the dissolution of at least portions of the anchor element, for example by virtue of the entire anchor element being dissolved galvanically or merely a portion of the anchor element being dissolved galvanically. As a result of the galvanically releasable anchorage, it is possible to manage without alternatively conceivable mechanisms, actuators or other devices for releasing the anchor element. It is understood that a plurality of anchor elements designed according to the present disclosure may also be present. In principle, the anchor element itself may have any design suitable for the present purpose. In one embodiment, the anchor element is configured for interlocking anchorage to the body tissue. In an alternative to that or in addition, a further embodiment of the anchor element is configured for frictionally connected anchorage to the body tissue. In one embodiment, the at least one stimulation electrode is formed on the catheter shaft. In other words: The catheter shaft itself, as it were, forms the at least one stimulation electrode. In a further embodiment, the stimulation electrode is longitudinally extended and, as it were, forms the catheter shaft.

In an embodiment of the present disclosure, the entire anchor element is manufactured from the galvanic material. Consequently, the entire anchor element is galvanically dissolved under the action of the DC voltage signal. In comparison with merely portions of the anchor element being galvanically dissolved, this avoids undissolved constituents of the anchor element remaining in the patient's body. In addition, comparatively simple manufacture is achieved since the entire anchor element may be manufactured from the same material.

In a further embodiment of the present disclosure, the anchor element comprises an anchor portion and a securing portion. The anchor portion is configured to interact with the body tissue. In other words: The anchor portion serves the actual interlocking and/or frictionally connected anchorage to the body tissue. The securing portion serves to secure the anchor element to the catheter shaft. The securing portion is manufactured from the galvanic material, and the anchor portion is manufactured from a resorbable material. Consequently, only the securing portion is galvanically dissolved under the action of the DC voltage signal. As a result, the remainder of the anchor element, specifically the anchor portion, is separated from the catheter shaft. The anchor portion itself is not dissolved galvanically in the process and initially remains in the patient's body. Since the anchor portion is manufactured from the aforementioned resorbable material, it is taken up/dissolved (resorbed) by the patient's body after a certain period of time. In principle, all resorbable materials suitable for the present purpose are considered. Such resorbable materials are known to the person skilled in the relevant art and therefore need not be specified in detail at this point. By manufacturing only the securing portion from the galvanic material, excessive accumulation of dissolved material in the patient's body may be avoided. This is particularly true in comparison with the anchor element being completely manufactured from the galvanic material.

In a further embodiment of the present disclosure, the galvanic material includes iron, nickel, tin, zinc and/or copper. In an alternative to that, the galvanic material is iron, nickel, tin, zinc and/or copper. The aforementioned materials occur in the human body as trace elements. Therefore, no health impairments are to be feared on account of a dissolution of the aforementioned galvanic materials in the patient's body, provided that the amount of material used for the anchor element and consequently a concentration arising after the galvanic dissolution in the patient's body observes certain limit values. The use of zinc has proven to be particularly advantageous.

In a further embodiment of the present disclosure, the anchor element is a longitudinally extended helix, which is configured to be screwed into the body tissue. To anchor the catheter shaft, the helix is rotated about its longitudinal axis and thereby screwed into the body tissue. By preference, the helix is arranged at a distal end face of the catheter shaft and protrudes coaxially from the catheter shaft in the distal direction. Consequently, the entire catheter shaft may be rotated about its longitudinal axis in order to screw in the helix. In one embodiment, the entire helix is manufactured from the galvanically dissolvable material. In a further embodiment, only a proximal end of the helix, by means of which the helix is secured to the catheter shaft, is manufactured from the galvanically dissolvable material.

In a further embodiment of the present disclosure, the anchor element is a barbed element, which is configured to be hooked into the body tissue. The barbed element counteracts proximally directed movement of the catheter shaft. As a result, the barbed element prevents the catheter shaft from being unintentionally pulled out of the body tissue. This does not preclude the barbed element from also counteracting a proximal feed of the catheter shaft. In one embodiment, the entire barbed element is manufactured from the galvanically dissolvable material. In a further embodiment, merely a securing portion of the barbed element is manufactured from the galvanic material, with the securing portion being attached directly to the catheter shaft.

In a further embodiment of the present disclosure, the barbed element is displaceable relative to the catheter shaft between an insertion position and an anchor position, wherein the barbed element rests against the catheter shaft in the insertion position and is splayed away from the catheter shaft in the anchor position. Such displaceability of the anchor element between the insertion position and the anchor position enables a simplified insertion of the catheter shaft into the body tissue. For this purpose, the barbed element rests against the catheter shaft in the insertion position. In other words: The barbed element rests tightly against the catheter shaft. In the anchor position, the barbed element is splayed away from the catheter shaft. Splaying out the barbed element initiates the actual barb function thereof. In other words: In the anchor position, the barbed element protrudes outward from the catheter shaft and may thereby hook into the body tissue in frictionally connected and/or interlocking fashion. In one embodiment, the displacement element is rotationally movable relative to the catheter shaft between the insertion position and the anchor position. In a further embodiment, a pivoting movement is provided. A pivot bearing is provided in one embodiment for the corresponding movable bearing of the anchor element. A swivel bearing, which may be designed as a flexure in particular, is provided in a further embodiment.

In a further embodiment of the present disclosure, the barbed element is prestressed by means of a prestressing element in the direction of the anchor position starting from the insertion position and is affixed in the insertion position by means of a biocompatible adhesive, the biocompatible adhesive being soluble under the action of a physical and/or chemical property of the body tissue. This embodiment of the present disclosure allows a simplified insertion of the catheter and its automated anchorage after insertion. For this purpose, the at least one barbed element is affixed in the insertion position by means of the biocompatible adhesive. This prevents the barbed element from being unintentionally displaced into the anchor position during the catheter arrangement. After the catheter arrangement/insertion of the catheter shaft, the biocompatible adhesive is exposed to the action of the body tissue. In order to enable an automated release of the affixment, the biocompatible adhesive is configured such that it is dissolvable under the action of the aforementioned physical and/or chemical property of the body tissue. After the biocompatible adhesive has dissolved, the prestress of the barbed element in the direction of the anchor position causes automated formation of the anchorage. In principle, the prestressing element may have any design suitable for the present purpose in this case. For example, the prestressing element may be formed by an elastic portion of the catheter shaft and/or of the anchor element. In an alternative to that or in addition, the prestressing element may be embodied as an additional component, in particular as a spring element, with different constructions being conceivable (spiral spring, torsion spring, leg spring or the like).

In further embodiment of the present disclosure, the property is a moisture, fat, a temperature and/or a pH value. Consequently, the biocompatible adhesive is soluble under the action of body tissue moisture, body fat, body temperature and/or a pH value of body tissue. Correspondingly configured biocompatible materials are known as a matter of principle to a specialist working in the field of medical bonding technology and therefore need not be specified in all details at this point.

In a further embodiment of the present disclosure, the biocompatible adhesive contains sugar and/or starch. In a preferred embodiment of the present disclosure, the biocompatible adhesive is sugar, in particular formed from sugar molecules. This renders the biocompatible adhesive dissolvable under the action of the moisture of the body tissue in a particularly simple and reliable manner. In addition, there is no risk of any health impairment due to the adhesive dissolved as sugar.

The present disclosure also relates to a medical system having a neurostimulation catheter according to the preceding description and having a signal generator. The signal generator is connected to the anchor element and configured to create the DC voltage signal. The connection between the signal generator and the at least one anchor element serves the transmission of the DC voltage signal for galvanically dissolving at least portions of the anchor element. In one embodiment, this connection is a wired connection. In a further embodiment, a wireless connection is provided.

In a further embodiment of the present disclosure, the signal generator is connected to the at least one stimulation electrode and configured to create a DC voltage-free stimulation signal. The stimulation signal is configured to inhibit the transmission of excitation of the nerve to be numbed. In this embodiment, the signal generator thus has a dual function and serves firstly to create the DC voltage signal and secondly to create the electric stimulation signal. Since the stimulation signal is DC voltage-free, i.e. has no DC components, an unwanted superposition on the DC voltage signal is avoided. In particular, this avoids the situation where the galvanic material of the anchor element unintentionally dissolves under the action of the stimulation signal and thus leads to the anchorage being removed prematurely.

The present disclosure also relates to a method for anchoring and releasing a neurostimulation catheter. The method according to the present disclosure comprises the steps of: anchoring a catheter shaft of the neurostimulation catheter, the catheter shaft being anchored to a body tissue, which surrounds the catheter shaft, by means of an anchor element secured to the catheter shaft; releasing the anchorage, with a DC voltage signal being applied to the anchor element, at least one portion of the anchor element manufactured from a galvanic material being galvanically dissolved under the action of the DC voltage signal and hence the anchorage to the body tissue being released. The advantages associated with the method according to the present disclosure correspond with the advantages of the neurostimulation catheter according to the present disclosure. Further embodiments of the method according to the present disclosure emerge from the features of the embodiments of the neurostimulation catheter according to the present disclosure and of the medical system. To avoid repetition, reference is therefore made to the relevant disclosure and explicit reference is made.

### BRIEF DESCRIPTION OF THE DRAWINGS

Further advantages and features of the present disclosure will emerge from the claims and from the following description of preferred exemplary embodiments of the present disclosure, which are illustrated with the aid of the drawings.

FIG. 1 shows, in schematically simplified illustration, an embodiment of a medical system according to the present disclosure having a neurostimulation catheter and a signal generator, the neurostimulation catheter being provided with anchor elements;

FIG. 2 shows a distal end of the neurostimulation catheter, in further simplified illustration, and with splayed-out anchor elements for anchorage purposes;

FIG. 3 shows the distal end of the neurostimulation catheter after galvanic dissolution of the anchor elements;

FIGS. 4 and 5 each show a distal end of a further embodiment of a neurostimulation catheter according to the present disclosure, in which only portions of the anchor elements are in each case manufactured from a galvanic material;

FIGS. 6 and 7 each show a distal end of a further embodiment of a neurostimulation catheter according to the present disclosure, in which the anchor elements are affixed by means of a biocompatible adhesive in an abutting insertion position (FIG. 6) and prestressed by means of a prestressing element in a splayed anchor position (FIG. 7);

FIGS. 8 and 9 each show a distal end of a further embodiment of a neurostimulation catheter according to the present disclosure, wherein an anchor element is designed as a helix and is completely manufactured from a galvanically dissolvable material;

FIGS. 10 and 11 each show a distal end of a further embodiment of a neurostimulation catheter according to the present disclosure, wherein the anchor element is once again manufactured as a helix but only portions thereof are manufactured from a galvanic material; and

FIG. 12 shows a schematic block representation of an embodiment of a method according to the present disclosure for anchoring and releasing a neurostimulation catheter.

### DETAILED DESCRIPTION

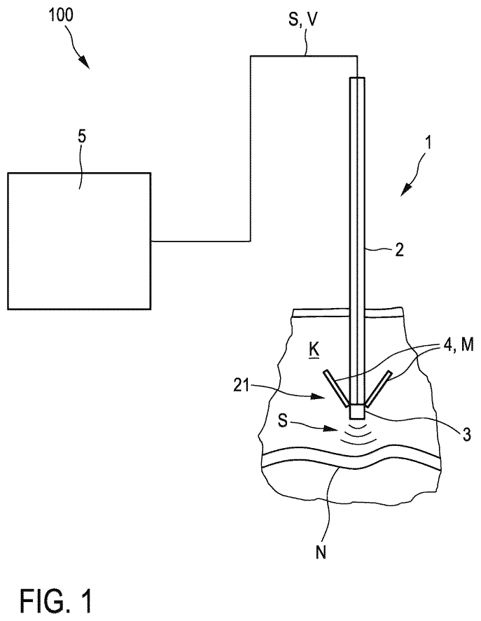

According to FIG. 1, a medical system 100 is provided for use in pain therapy by means of electrical neurostimulation. In FIG. 1, the medical system 100 is depicted in an exemplary use situation in a schematically highly simplified manner.

The medical system 100 comprises a neurostimulation catheter 1 with a longitudinally extended catheter shaft 2 and at least one stimulation electrode 3 arranged on the catheter shaft 2. In addition, the medical system 100 comprises a signal generator 5, which is configured to create the electrical stimulation signal S.

In the exemplary use situation shown, the catheter shaft 2 is inserted into a body tissue K of a patient. In this case, the stimulation electrode 3 is arranged in the vicinity of a nerve N. The stimulation signal S, which can be created by means of the signal generator 5 and output by means of the stimulation electrode 3, is configured to inhibit an excitation transmission of the nerve N. This inhibition suppresses the patient's sense of pain. The effectiveness of the stimulation signal S is decisively influenced by the positioning of the stimulation electrode 3 in relation to the nerve N. In particular, a proximal dislocation of the catheter shaft 2 and hence of the stimulation electrode 3 may lead to an excessive increase in the distance between the stimulation electrode 3 and the nerve N and hence to an impairment of the pain suppression. Incidentally, such pain therapy via neurostimulation or neuromodulation is known in principle to a person skilled in the relevant art. Further details on this therefore need not be discussed at this point.

In order to avoid an unwanted dislocation of the stimulation electrode 3, the neurostimulation catheter 1 comprises at least one anchor element 4 that is secured to the catheter shaft 2.

Two anchor elements 4 are present in the embodiment shown. In further embodiments, only one (single) anchor element is present, or else there are more anchor elements than the two anchor elements present here. For the sake of the required brevity, reference is only made to a/the anchor element 4 below. The relevant disclosure also applies to the further (second) anchor element and any other anchor elements.

The anchor element 4 is configured to anchor to the body tissue K. In this context, FIG. 1 shows such an anchoring by way of example. For anchorage purposes, the anchor element 4 interacts with the surrounding body tissue K in frictionally connected and/or interlocking fashion. In particular, this counteracts a proximal displacement of the catheter shaft 2.

The neurostimulation catheter 1 must be removed from the body tissue K following the completion of the actual pain therapy. For this purpose, the catheter shaft 2 is pulled proximally out of the body tissue K together with the stimulation electrode 3. The anchorage by means of the anchor element 4 must first be released. For this purpose, at least a portion of the anchor element 4 is manufactured from a galvanic material M. The galvanic material M can be galvanically dissolved under the action of a DC voltage signal V. By dissolving the galvanic material M, the anchorage between the anchor element 4 and the body tissue K is released.

In the embodiment shown, the signal generator 5 is configured to create the DC voltage signal S and connected to the anchor element 4. Said connection is established via a signal line (without reference sign), which may be wired or wireless. In the embodiment shown, the stimulation signal S is also transmitted via said signal line.

In addition, the stimulation electrode 3 and the anchor element 4 are arranged at a distal end 21 of the catheter shaft 2 in the embodiment shown. The catheter shaft is longitudinally extended between the distal end 21 and a proximal end (without reference sign), which is arranged outside the body tissue K in the exemplary use situation shown.

The positioning of the anchor element 4, as shown in the figures, at the distal end 21 and in relation to the stimulation electrode 3 should be understood as purely exemplary and not to scale. In order to avoid mechanical irritation of the nerve N by the anchor element 4, it may be advantageous to arrange the anchor element 4 on the catheter shaft 2 in a manner offset further than the stimulation electrode 3 in the proximal direction, as shown schematically in the figures.

The catheter shaft 2 may also be referred to as a stimulator shaft. In the embodiment shown in the figures, the stimulation electrode 3 is formed on the catheter shaft 2. In other words: The catheter shaft itself forms the stimulation electrode. Conversely, it can also be said that the stimulation electrode is longitudinally extended and, as it were, forms the catheter shaft/stimulator shaft. In the simplest case, catheter shaft and stimulation electrode are formed by a wire.

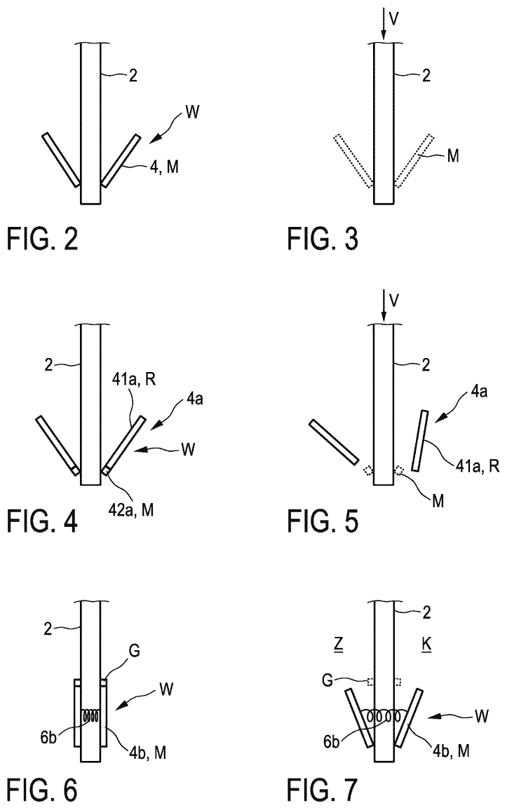

The distal end 21 is shown in detail in FIGS. 2 and 3, but the stimulation electrode 3 is masked in said figures. In this case, FIG. 2 shows an anchor position with an (as yet) undissolved anchor element 4. In FIG. 3, the anchor element 4 is galvanically dissolved under the action of the DC voltage signal V. This is symbolically illustrated in FIG. 3 by the dotted contour line.

In the embodiment shown, the galvanic material M is zinc. In embodiments not shown in the drawings, the galvanic material is iron, nickel, tin and/or copper.

In the embodiment shown in FIGS. 1 to 3, the entire anchor element 4 is manufactured from the galvanic material M. Consequently, the entire anchor element 4 is galvanically dissolved under the action of the DC voltage signal V.

By contrast, FIGS. 4 and 5 show an embodiment with an anchor element 4a, only portions of which are manufactured from the galvanic material M. In particular, the anchor element 4a comprises an anchor portion 41a and a securing portion 42a. The securing portion 42a is secured directly to the catheter shaft 2. The anchor portion 41a serves for the actual anchorage to the body tissue K and is securely connected to the securing portion 42a. The securing portion 42a is significantly smaller than the anchor portion 41a. The securing portion 42a is manufactured from the galvanic material M. The anchor portion 41a is manufactured from a resorbable material R. For example, the resorbable material R might be a resorbable plastic or the like. Consequently, only the securing portion 42a is dissolved under the action of the DC voltage signal V (see FIG. 5). As a result, the anchor portion 41a, i.e. the rest of the anchor element 4a, is separated from the catheter shaft 2 and initially remains in the body tissue K. As a result of the manufacture from the aforementioned resorbable material R, the anchor portion 41a is dissolved/resorbed in the body tissue K over time.

In the embodiment according to FIGS. 1 to 3 and in the embodiment of FIGS. 4 and 5, the anchor element 4 and 4a is a barbed element W in each case. The barbed element W splays outward away from the catheter shaft 2 in the anchor position (FIGS. 1, 2, 4). In other words: The barbed element W protrudes from the catheter shaft 2 for anchorage to the body tissue K.

The anchor element, in particular the barbed element, may be immobile or movable relative to the catheter shaft. In this context, FIGS. 6 and 7 show an embodiment with a relatively movable anchor element.

In the aforementioned embodiment according to FIGS. 6 and 7, the anchor element 4b is also designed as a barbed element W. The barbed element W is displaceable relative to the catheter shaft 2 between an insertion position (FIG. 6) and an anchor position (FIG. 7). In the insertion position, the barbed element W rests against the catheter shaft 2. In the anchor position, the barbed element is splayed away from the catheter shaft 2. In this case, the barbed element W is elastically prestressed by means of a prestressing element 6b in the direction of the anchor position starting from the insertion position. In addition, the barbed element W is affixed in the insertion position. A biocompatible adhesive G is present for affixment purposes. Its position shown in FIGS. 6 and 7 should be understood as purely exemplary. The same applies to the position and design of the prestressing element, which is shown as a helix in exemplary fashion in FIGS. 6 and 7.

The biocompatible adhesive G is soluble under the action of the body tissue K, in particular under the action of a property Z of the body tissue. The property Z is a physical and/or chemical property, for example a moisture, a temperature and/or a pH value of the body tissue K. In the embodiment shown, the biocompatible adhesive G is soluble under the action of the moisture of the body tissue K. The moisture of the body tissue K will dissolve the adhesive G as soon as the catheter shaft 2 is inserted into said body tissue together with the anchor element 4b, which is affixed in the insertion position by adhesive G. After dissolution of the adhesive G (see FIG. 7), the prestressing element 6b causes an automated displacement of the anchor element 4b in the direction of the anchor position. The anchorage is released as described with reference to FIGS. 1 to 3 or 4 and 5. In this case, the anchor element 4b may in turn be manufactured from the galvanic material M in full or only in the region of a securing portion.

In the embodiment shown, the biocompatible adhesive G is a sugar, in particular formed from sugar molecules.

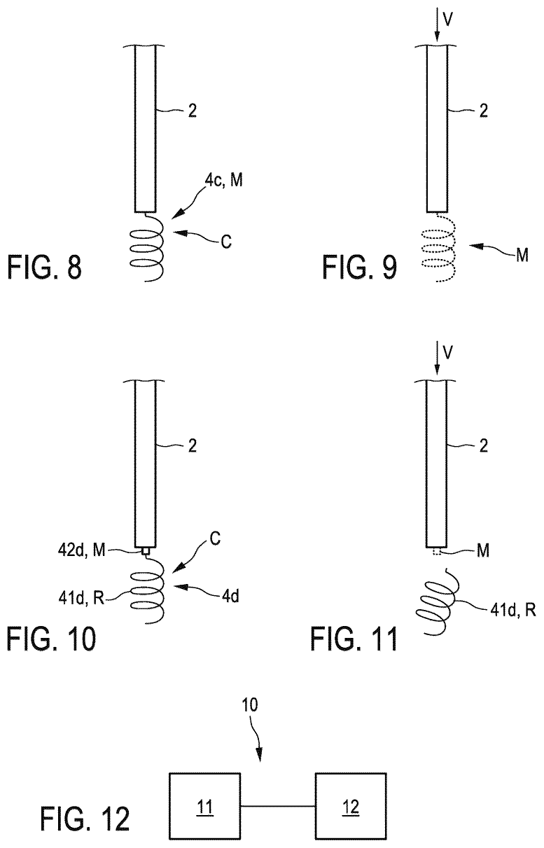

In the embodiments according to FIGS. 8 and 9 and FIGS. 10 and 11, the respective anchor elements 4c, 4d are designed as a helix C. Starting from the distal end of the catheter shaft 2, the helix C extends further in the distal direction and is configured to be screwed into the body tissue K. It is understood that the shaping of the helices C shown in FIGS. 8 to 11 and their relative dimensions in comparison with the catheter shaft 2 should be understood as purely exemplary.

In the embodiment according to FIGS. 8 and 9, the entire anchor element 4c, i.e. the entire helix C, is manufactured from the galvanic material M. The entire helix C dissolves under the action of the DC voltage signal V (see FIG. 9).

By contrast, only at least one portion of the anchor element 4d is manufactured from the galvanically dissolvable material M in the embodiment according to FIGS. 10 and 11. In this case, the anchor element 4d in the form of the helix C in turn comprises a securing portion 42d and an anchor portion 41d. The anchor portion 41d forms at least the majority of the helix C. The securing portion 42d is arranged at one end of the helix C and directly secured to the distal end of the catheter shaft 2. Once again, the anchor portion 41d is manufactured from the resorbable material R. Only the securing portion 42d is galvanically dissolved under the action of the DC voltage signal V, whereby the anchor portion 41d is separated from the catheter shaft 2.

In FIG. 12, an embodiment of a method according to the present disclosure for anchoring and releasing a neurostimulation catheter according to the preceding embodiments is schematically illustrated in a highly simplified manner. The method 10 provides for anchoring 11 of the catheter shaft, wherein the catheter shaft is anchored to the body tissue by means of the anchor element which is secured to the catheter shaft. Further, the method 10 provides for releasing 12 of the anchorage according to the preceding description in relation to the embodiments according to FIGS. 1 to 11. Accordingly, the DC voltage signal V is applied to the anchor element, whereby the latter is dissolved at least in part (as in the embodiments according to FIGS. 4, 5, 10 and 11) or in full (as in the embodiments of FIGS. 1 to 3 and 8 and 9), and the anchorage to the body tissue K is released.

1. A neurostimulation catheter for use in pain therapy, the neurostimulation catheter comprising:
a catheter shaft that is longitudinally extended;
at least one stimulation electrode arranged on the catheter shaft and configured to deliver an electrical stimulation signal; and
at least one anchor element secured to the catheter shaft,
the at least one anchor element configured to be anchored to a body tissue that surrounds the catheter shaft, and
the at least one anchor element comprising one or more portions manufactured from a galvanic material that are galvanically dissolvable under action of a DC voltage signal to release anchorage of the at least one anchor element from the body tissue.

2. The neurostimulation catheter according to claim 1, wherein the one or more portions of the at least one anchor element comprise all of the at least one anchor element.

3. The neurostimulation catheter according to claim 1, wherein the at least one anchor element comprises an anchor portion configured to interact with the body tissue, and a securing portion by which the at least one anchor element is secured to the catheter shaft, with the securing portion being manufactured from the galvanic material and the anchor portion being manufactured from a resorbable material.

4. The neurostimulation catheter according to claim 1, wherein the galvanic material contains or is iron.

5. The neurostimulation catheter according to claim 1, wherein the galvanic material contains or is nickel.

6. The neurostimulation catheter according to claim 1, wherein the galvanic material contains or is tin.

7. The neurostimulation catheter according to claim 1, wherein the galvanic material contains or is copper.

8. The neurostimulation catheter according to claim 1, wherein the galvanic material contains or is zinc.

9. The neurostimulation catheter according to claim 1, wherein the at least one anchor element is a longitudinally extended helix configured to be screwed into the body tissue.

10. The neurostimulation catheter according to claim 1, wherein the at least one anchor element is a barbed element configured to be hooked into the body tissue.

11. The neurostimulation catheter according to claim 10, wherein:
the barbed element is displaceable relative to the catheter shaft between an insertion position and an anchor position, and
the barbed element rests against the catheter shaft in the insertion position and is splayed away from the catheter shaft in the anchor position.

12. The neurostimulation catheter according to claim 11, wherein the barbed element is prestressed by a prestressing element in a direction of the anchor position starting from the insertion position and is affixed in the insertion position by a biocompatible adhesive, the biocompatible adhesive being soluble under action of a physical and/or chemical property of the body tissue.

13. The neurostimulation catheter according to claim 12, wherein the physical and/or chemical property is moisture, fat, temperature and/or a pH value.

14. The neurostimulation catheter according to claim 12, wherein the biocompatible adhesive contains or is sugar and/or starch.

15. A medical system comprising:
the neurostimulation catheter according to claim 1; and
a signal generator connected to the at least one anchor element and configured to create the DC voltage signal.

16. The medical system according to claim 15, wherein the signal generator is connected to the at least one stimulation electrode and configured to create a DC voltage-free stimulation signal.

17. A method for anchoring and releasing a neurostimulation catheter, the method comprising the steps of:
anchoring a catheter shaft of the neurostimulation catheter to a body tissue, the catheter shaft being anchored to the body tissue that surrounds the catheter shaft by means of an anchor element secured to the catheter shaft; and
releasing the catheter shaft from the body tissue with a DC voltage signal applied to the anchor element,
at least one portion of the anchor element being manufactured from a galvanic material that is galvanically dissolved under action of the DC voltage signal to release the catheter shaft from the body tissue.
